(19) United States
(12) Patent Application Publication
(10) Pub. No.: US 2025/0060353 A1
(43) Pub. Date: Feb. 20, 2025

(54) NANOFABRICATED SEQUENCING DEVICES WITH DETERMINISTIC MEMBRANE APERTURES BORDERED BY ELECTROMAGNETIC FIELD ENHANCEMENT ANTENNAS

(71) Applicant: LSPR AG, Wallisellen (CH)

(72) Inventors: Benedikt OSWALD, Wallisellen (CH); Peter WARNICKE, Wallisellen (CH)

(73) Assignee: LSPR AG, Wallisellen (CH)

(21) Appl. No.: 18/722,454

(22) PCT Filed: Dec. 22, 2021
(86) PCT No.: PCT/EP2021/087285

§ 371 Date: Jun. 20, 2024

(51) Int. Cl. G01N 33/487; B82Y 15/00; G01N 21/65

(52) U.S. Cl. CPC G01N 33/48721; G01N 21/658; B82Y 15/00

(57) ABSTRACT

The invention is notably directed to an optical sensing device (1, 1a). The device has a layer structure comprising a substrate (10), a dielectric layer (11), and opposite antenna elements (17). The substrate is structured to laterally delimit a cavity. The dielectric layer extends on top of the substrate and forms a membrane spanning the cavity. The membrane including n apertures (i.e., nanopores) to the cavity, where n≥1. Two or more apertures (30) are preferably provided (n≥2). More preferably, the number of apertures is larger than or equal to 100 or, even, 400. There are n pairs of opposite antenna elements, which are patterned on top of the dielectric layer, on opposite lateral sides of respective ones of the n apertures. The n pairs of opposite antenna elements define n respective gaps extending between opposite antenna elements of the n pairs along respective directions parallel to a main plane of the substrate. The n pairs of opposite antenna elements define, together with the respective apertures, n molecular passages. Each passage extends from the cavity through a respective one of the n apertures (i.e., through the membrane) and a respective one of the n gaps, along a direction transverse to the main plane of the substrate. The average length of the n gaps along said respective directions is between 4 nm and 20 nm. The n gaps define respective electromagnetic field enhancement regions, in which electromagnetic radiation can be concentrated upon irradiating the antenna elements, for optically sensing molecules, in operation. Moreover, the average diameter of the n apertures is larger than or equal to the average length of the gaps along their respective directions. Thus, the minimal cross-sectional dimension of each of the n passages is limited by a respective one of the n gaps along said respective directions. The invention is further directed to related apparatuses, sensing methods, and fabrication methods.

### CLAIM FOR PRIORITY

The present application is a national stage filing under 35 U.S.C 371 of PCT application number PCT/EP2021/087285, having an international filing date of Dec. 22, 2021, the disclosure of which is hereby incorporated by reference in its entirety.

### BACKGROUND

The invention relates in general to the field of nanofabricated optical sensing devices, optical sensing apparatuses, methods of fabrication of such optical sensing devices, and methods for optically sensing analytes (such as DNA and RNA molecules). In particular, it is directed to nanofabricated sequencing devices, in which apertures are deterministically defined in membranes, where the apertures are bordered by pairs of electromagnetic field enhancement antenna elements, to define passages in which analyte molecules can be controllably trapped for sensing, e.g., using surface-enhanced Raman spectroscopy techniques. The invention notably provides a practical implementation of plasmonic nanopore single-molecule DNA sequencing.

The following documents forms part of the background art:
“Plasmonic Nanopores for Trapping, Controlling Displacement, and Sequencing of DNA”. Maxim Belkin, Shu-Han Chao, Magnus P. Jonsson, Cees Dekker, and Aleksei Aksimentiev. ACS Nano 2015 9 (11), 10598-10611. DOI: 10.1021/acsnano.5b04173; and
Patent Application WO2020150140A1 (Armonica Technologies, Inc., hereafter referred to as “Armonica”).

Belkin et al. have theorized the concept of plasmonic, solid-state nanopore DNA sequencing. I.e., the authors have theoretically investigated and numerically analyzed a DNA sequencing methodology, where nanoplasmonics are used to control the translocation of a DNA molecule through a solid-state nanopore. That is, the authors propose to use plasmonic trapping, i.e., catching and holding a DNA strand when it tunnels through a nanopore formed in a membrane, while surface-enhanced Raman spectroscopy is used to obtain the sequence information. The aperture is assumed to be neighbored on top by two gold triangular prisms, which form a bowtie structure on top of the solid-state membrane. The nanopore in the gap of the bowtie structure connects one side of the membrane to the other. The entire structure is assumed to be immersed in an electrolyte solution and a transmembrane potential is applied to cause DNA molecules to travel from one side of the membrane to the other through the nanopore. The bowtie structure is used to focus an incident laser beam onto a nanometer-size hot spot in proximity of the nanopore. The optical field of the hot spots gives rise to a restraining force on the DNA molecule, which force counteracts the pull of the electrophoretic force. Switching the laser beam on and off results in stepwise displacement of the DNA molecule through the nanopore. Surface-enhanced Raman scattering (SERS) is used to characterize the nucleotide composition of the DNA fragment confined within the hot spots. The nucleotide sequence of DNA can then be deciphered through deconvolution of the SERS signals at the frequencies that uniquely identify each of the four DNA nucleotides, the authors say. As interesting as this approach may be, and notwithstanding its theoretical merits, the required nanostructure has not been experimentally demonstrated so far. This is manifestly due to the formidable challenge of processing antenna elements bordering a nanopore connecting two opposite sides of a thin membrane.

According to the present inventors, the known lithographic methods do not allow to reproducibly obtain such a nanostructure with sufficient precision, which may explain why alternative approaches are being explored, experimentally speaking.

For instance, an alternative approach is proposed in the patent application WO2020150140A1 (to Armonica), which proposes a DNA sequencing technology relying on tortuous nanopores, to identify nucleotides thanks to Raman spectroscopy once these nucleotides appear at the opening of the tortuous nanopore. The nanopores are obtained thanks to a randomized self-assembly process, which precludes a controlled fabrication of predetermined nanostructures, aligned with solid-state nanopores of determined shape, geometry, size, and position. As a result, and contrary to Belkin's suggestion, WO2020150140A1 does not rely on optical trapping in order to control the motion of the DNA strands tunnelling through the nanopores. Rather, this is the tortuous structure of the nanopores that makes is possible to decelerate the tunnelling of DNA strands. Deceleration is necessary in order to be able to measure the Raman spectrum of individual nucleotides. The advantage of this approach is that it greatly reduces the difficulty of the nanofabrication process because no structures of predetermined shape, geometry, size, and materialization, need to be fabricated. A disadvantage, however, is that the self-assembly (or random build-up) of the nanopores lead to situations where it is not clear how to keep control on the translocation of DNA molecules.

It is the opinion of the present inventors that the tortuous nanopores preclude a precise alignment of plasmonic nanostructures with the nanopore openings, such that the efficiency of SERS excitation and the subsequent readout process may be substantially reduced. In addition, this approach complicates the design of a spectrometer. I.e., the efficiency of Raman measurements is jeopardized by the fact that it is, a priori, unclear from which spatial positions the Raman spectra must be read.

Thus, the present inventors came to the conclusion that there is a need for a nanofabrication process, which is capable of producing plasmonic structures with predetermined shape, geometry, location, materials, and size, in proximity with nanopores that are deterministically positioned and structured. Such a process should further make it possible to precisely control the size of the nanopores and their placements between the tips of the plasmonic structures with nanometer precision. The precision of the resulting structures may for instance be verified with a transmission electron microscope (TEM).

A plasmonic trapping as theoretically proposed by Belkin et al. requires a plasmonic (i.e., nano-optical) hotspot, i.e., an electromagnetic field enhancement region. A plasmonic hotspot is a region in the vicinity of a metallic nanoparticle, where the electric field can be enhanced. The purpose of such a plasmonic hotspot is two-fold. First, it serves to trap a DNA strand at a specific nucleotide and, second, it amplifies the Raman signal emitted by the nucleotide when illuminated by a laser. This Raman signal amplification is at the core of SERS. Without the SERS effect, it is not possible to measure the Raman spectrum of a single nucleotide since the associated Raman signal is too weak and cannot be distinguished from the background noise. Without plasmonic trapping, the DNA strand would tunnel through the nanopore with a speed that prevents a Raman spectrum to be recorded with sufficient accuracy, a priori.

Therefore, the problem is to be able to obtain a physical realization of a device allowing a sensing methodology such as proposed by Belkin et al. to be achieved, and to check whether the resulting concept works at all, given the lack of experimental results so far. All the more, what is needed is a fabrication methodology allowing such a device to be achieved, which is a priori not feasible.

### SUMMARY

According to a first aspect, the present invention is embodied as an optical sensing device. The device has a layer structure comprising a substrate, a dielectric layer, and opposite antenna elements. The substrate is structured to laterally delimit a cavity. The dielectric layer extends on top of the substrate and forms a membrane spanning the cavity. The membrane including n apertures (i.e., nanopores) to the cavity, where n≥1. Two or more apertures are preferably provided (n≥2). More preferably, the number of apertures is larger than or equal to 100 or, even, 400. There are n pairs of opposite antenna elements, which are patterned on top of the dielectric layer, on opposite lateral sides of respective ones of the n apertures. The n pairs of opposite antenna elements define n respective gaps extending between opposite antenna elements of the n pairs along respective directions parallel to a main plane of the substrate. The n pairs of opposite antenna elements define, together with the respective apertures, n molecular passages. Each passage extends from the cavity through a respective one of the n apertures (i.e., through the membrane) and a respective one of the n gaps, along a direction transverse to the main plane of the substrate. The average length of the n gaps along said respective directions is between 4 nm and 20 nm. The n gaps define respective electromagnetic field enhancement regions, in which electromagnetic radiation can be concentrated upon irradiating the antenna elements, for optically sensing molecules, in operation. Moreover, the average diameter of the n apertures is larger than or equal to the average length of the gaps along their respective directions. Thus, the minimal cross-sectional dimension of each of the n passages is limited by a respective one of the n gaps along said respective directions.

According to the present design, the antenna elements can be substantially flush with, or even hang over the edges of the aperture underneath. However, the antenna elements will not be laterally recessed with respect to the edges of this aperture, subject to fabrication tolerances on the order of the nm, according to TEM imaging. This means that the minimal cross-sectional dimension of each passage (as defined by a corresponding aperture in the dielectric layer) is actually limited by the gap between opposite antenna elements bordering this aperture. This allows the electromagnetic field enhancement volume defined between the opposite antenna elements to be minimized, for a given cross-sectional dimension of the corresponding aperture. This, in turn, allows the electromagnetic field enhancement to be maximized, all things being otherwise equal.

On the contrary, the existing nanofabrication methods would likely require the patterned antenna elements to be laterally recessed with respect to the edges of the corresponding aperture. In that respect, a device configuration as described above is extremely challenging to process with accuracy tolerance on the order of the nm, because the gaps between antenna elements are defined above respective apertures defined in a thin membrane. The skilled person will appreciate that the fabrication process is, in that case, much more challenging, a priori, than in a context where the antenna elements lay flat on a thick substrate. One reason is that the antenna elements and the apertures require distinct process steps, which are, a priori, incompatible. I.e., one process step may likely cause to damage the result of the other processing step, as discussed in detail in the next section.

Fortunately, the present description discloses fabrication methods that resolve this incompatibility and further make it possible to achieve nanostructures with a satisfactory accuracy, i.e., on the order of the nm. More generally, the present fabrication methods allow nanostructures to be obtained with deterministic and reproducible shapes, dimensions, and positions, unlike prior fabrication methods.

In embodiments, in particular related to sequencing chips, the number n of the apertures is larger than or equal to 100, and preferably larger than or equal to 400. Such a chip may for instance aggregate several membranes (side-by-side) with respective cavities, which results in multiplying the number of apertures. For example, the present inventors have fabricated chips including nine membranes, each including 400 nanopores with deterministic shapes, dimensions, and locations.

Preferably, the average diameter of the n apertures is equal to the average length of the gaps, subject to ±3 nm, as verified by TEM imaging. That is, inner ends of the antenna elements of each of the n pairs are substantially flush with inner walls of the respective n apertures in the membrane.

Preferably, the diameters of the apertures and the lengths of the gaps are essentially constant, subject to a dispersion (i.e., a standard deviation) of less than (or equal to) 3 nm, as verified by TEM imaging.

In embodiments, the average, in-plane separation distance between the apertures is between 1 and 10 microns, preferably between 2 and 7 microns.

In preferred embodiments, the device includes two dielectric layers. I.e., the above dielectric layer is a first dielectric layer and the device further includes a second dielectric layer. The substrate is on top of the second dielectric layer. The substrate and the second dielectric layer are jointly structured to form a recess delimiting said cavity. For example, the substrate may comprise silicon, each of the two dielectric layers may comprise Si₃N₄, while the antenna elements may essentially comprise Au. In variants, the antenna elements comprise any other metal that exhibits plasmonic behavior in the desired wavelength region, e.g., in the range between 200 and 3000 nm, preferably between 700 and 1000 nm. The thickness of each of the first and second dielectric layers is preferably between 10 and 60 nm, and more preferably between 15 and 35 nm. Despite their low thicknesses, such dielectric layers were found to be surprisingly stable, mechanically and chemically speaking.

In embodiments, the optical sensing device further comprises one or more pairs of electrodes. The electrodes of each pair are on opposite sides of the membrane (i.e., the first dielectric layer). In variants, such electrodes do not form part of the device but are brough in contact with a liquid in which the device is immersed.

According to another aspect, the invention is embodied as an optical sensing apparatus. The apparatus notably comprises an optical sensing device as described above. It further includes a distributed electromagnetic source (e.g., comprising a Laser), which is configured to irradiate the antenna elements of each of the n pairs of opposite antenna elements. This way, electromagnetic radiation is concentrated in the respective electromagnetic field enhancement regions, with a view to optically sensing molecules in the respective gaps. The apparatus further includes a detector, which is configured to optically detect optical signals as modulated and/or generated by the molecules in said regions, in operation. The detector is preferably a Raman spectrometer. It may for instance be a spatial heterodyne Raman spectrometer.

In embodiments, the apparatus further comprises an electrical circuit comprising one or more pairs of electrodes, wherein the electrodes of each of the pairs are on opposite sides of the first dielectric layer. The electrical circuit is notably configured to apply a voltage bias between electrodes of each of the pairs to urge molecules through the passages. The optical sensing device of the apparatus may notably include two dielectric layers as described above, where the substrate extends on top of the second dielectric layer, so as to form a recess delimiting said cavity.

According to another aspect, the invention is embodied as a method of fabrication of an optical sensing device such as described above. The method comprises providing a substrate, depositing a dielectric layer on top of the substrate, and patterning fiducial marks on both the dielectric layer and the substrate. In addition, n pairs of opposite antenna elements are patterned on top of the dielectric layer (e.g., using electron beam lithography), based on an alignment protocol exploiting the fiducial marks previously patterned. This is done so as to define n respective gaps extending between opposite antenna elements of respective ones of the n pairs along respective directions parallel to a main plane of the substrate. Again, n≥1, preferably n≥2, and more preferably n≥100. The average length of the n gaps along their respective directions is between 4 nm and 20 nm. A protective layer is then deposited, so as to coat inner ends of the opposite antenna elements of each of the n pairs. The dielectric layer is subsequently dry etched (preferably using reactive-ion etching) at locations defined according to the fiducial marks, to open n apertures through the dielectric layer, between opposite antenna elements of respective ones of the n pairs. As a result, the opposite antenna elements of the n pairs are on opposite lateral sides of respective ones of the n apertures. Moreover, the antenna elements and the dielectric layer are coated with a protective polymer, for it to plug the gaps and the apertures. Next, the substrate is structured to form a recess extending up to the dielectric layer, so as for the latter to extend on top of residual, peripheral portions of the substrate and form a membrane spanning a cavity delimited by the recess. Eventually, the protective polymer is removed, to free up n molecular passages, each extending from the cavity through a respective one of the n apertures and a respective one of the n gaps along a direction transverse to the main plane of the substrate. The method is performed to obtain an optical sensing device, where the n gaps define respective electromagnetic field enhancement regions, in which electromagnetic radiation can be concentrated upon irradiating the antenna elements, for optically sensing molecules, in operation of the resulting device. Moreover, the average diameter of the n apertures is larger than or equal to the average length of the gaps along their respective directions, whereby a minimal cross-sectional dimension of each of the n passages is limited by a respective one of the n gaps along said respective directions.

The proposed method tackles the formidable challenge of achieving clean and precise nanostructures, where a pair of antenna elements are arranged on opposite lateral sides of nano-apertures formed through a membrane spanning a cavity, while ensuring deterministic and reproducible positions, shapes, and dimensions of the nanostructures. Basically, the proposed fabrication approach revolves around patterning the antenna elements (e.g., using electron beam lithography, or EBL for short), based on an alignment protocol exploiting previously patterned fiducial marks, prior to etching the apertures. However, the apertures are etched only after having protected inner ends of the antenna elements. The recess is then formed on the back side, but only after having coated the upper structures to protect the gaps and apertures. This way, the incompatibility noted earlier can be solved.

In preferred embodiments, the dielectric layer is a first dielectric layer, and the method further comprises depositing a second dielectric layer below the substrate, preferably while depositing the first dielectric layer. The second dielectric layer is patterned (after patterning the fiducial marks but prior to patterning the n pairs of opposite antenna elements) so as for residual, peripheral portions of the second dielectric layer to delimit the recess to be formed next.

Preferably, the fiducial marks are patterned using electron beam lithography and a dry etching procedure. The fiducial marks may for example be patterned as slits extending through the dielectric layer and partly in the substrate.

Preferably, the n pairs of opposite antenna elements are patterned as follows. First, a photoresist is deposited on top of the dielectric layer, for the photoresist to plug the slits. As a result, the photoresist protects the fiducial marks from being filled with metal (e.g., gold) during the subsequent deposition of the metallic layer. Second, the photoresist is structured by photolithography, for it to form residual plugs at a level of the slits. A metallic layer is then deposited on top of the dielectric layer and the residual plugs, and the residual plugs are then removed to define openings at the level of the slits (i.e., the fiducial marks). Next, an electron beam resist is deposited on top of the metallic layer. The electron beam resist is subsequently structured by electron beam lithography in accordance with shapes of the antenna elements. Finally, the metallic layer is etched through the structured electron beam resist using ion beam etching, to obtain the desired antenna elements.

In preferred embodiments, the protective layer is an alumina layer, which is obtained by coating an electron beam resist, opening cavities at the level of the gaps, and depositing the protective layer by atomic layer deposition at a temperature that is less than 90 C (to keep it compatible with materials in other layers). As a result, the protective layer notably coats the inner ends of the opposite antenna elements of each of the n pairs.

Preferably, the substrate essentially comprises silicon, each of the two dielectric layers essentially comprises Si₃N₄, and each of the antenna elements essentially comprises Au.

According to another aspect, the invention is embodied as a method for optically sensing an analyte. This method relies on an optical sensing device as described above. The method comprises irradiating the pairs of antenna elements of this device, to concentrate electromagnetic radiation in the electromagnetic field enhancement regions, and sensing molecules in the gaps by optically detecting optical signals that are modulated and/or generated by the molecules in the gaps, thanks to an optical detector, which preferably includes a Raman spectrometer, more preferably a spatial heterodyne Raman spectrometer. In particular, the present approach can be used to identify a nucleic acid sequence and can be applied to characterize both single and double stranded DNA sections.

Preferably, the sensing method further comprises applying an electric field across the membrane to urge molecules to the passages and trap the molecules at the gaps, by virtue of a combined effect of the electric field applied and the electromagnetic radiation concentrated in the respective electromagnetic field enhancement regions.

In preferred embodiments, the optical signals are detected with a Raman spectrometer, according to a surface-enhanced Raman spectroscopy technique, such as, surface enhanced Coherent anti-Stokes Raman spectroscopy (SECARS), surface-enhanced resonance Raman scattering (SERRS), surface enhanced hyper Raman scattering (SEHRS), or surface-enhanced Raman scattering (SERS). The molecules may notably comprise DNA or RNA molecules. In that case, the optical signals detected may be exploited to identify a nucleic acid sequence of the molecules, e.g., through the respective Raman fingerprint spectrum.

### BRIEF DESCRIPTION OF THE DRAWINGS

These and other objects, features and advantages of the present invention will become apparent from the following detailed description of illustrative embodiments thereof, which is to be read in connection with the accompanying drawings. The illustrations are for clarity in facilitating one skilled in the art in understanding the invention in conjunction with the detailed description. In the drawings:

FIGS. 1-31 are 2D cross-sectional views illustrating fabrication steps of an optical sensing device according to embodiments. Note, the device contains a single membrane with a single cavity in this example, for the sake of simplicity of the depiction. Moreover, the cross-sectional view depicted shows a single (row of) aperture(s), for simplicity. FIG. 31 shows the resulting structure, wherein the inner ends of the antenna elements are aligned (i.e., flush) with edges of the aperture (or nanopore) in this example;

FIG. 32 is a 3D view of an upper portion of the device of FIG. 31;

FIG. 33 is a 3D view of a device, where the membrane includes a 2D arrangement of apertures and corresponding antenna elements, according to embodiments;

FIG. 34A is a 3D view of a sensing apparatus including a device such as shown in FIG. 31 or 32, which further illustrates the operation of the device, as in embodiments;

FIG. 34B is a 2D cross-sectional view of the apparatus of FIG. 34A, further illustrating how a liquid containing DNA molecules can be spilled over the device, while the cavity underneath is filled with liquid, to trap and sense DNA molecules, as in embodiments;

FIG. 35 is a 2D cross-sectional view of a variant to the optical sensing device of FIG. 31, where the antenna elements partly hang over the aperture, as in embodiments; and

FIG. 36 is a corresponding 3D view.

The accompanying drawings show simplified representations of devices or parts thereof, as involved in embodiments. Technical features depicted in the drawings are not to scale. Similar or functionally similar elements in the figures have been allocated the same numeral references, unless otherwise indicated.

Devices, apparatuses, and methods embodying the present invention will now be described, by way of non-limiting examples.

### DETAILED DESCRIPTION OF EMBODIMENTS OF THE INVENTION

The following description is structured as follows. General embodiments and high-level variants are described in section 1. Section 2 addresses particularly preferred embodiments and applications, as well as technical implementation details.

### 1. General Embodiments and High-Level Variants

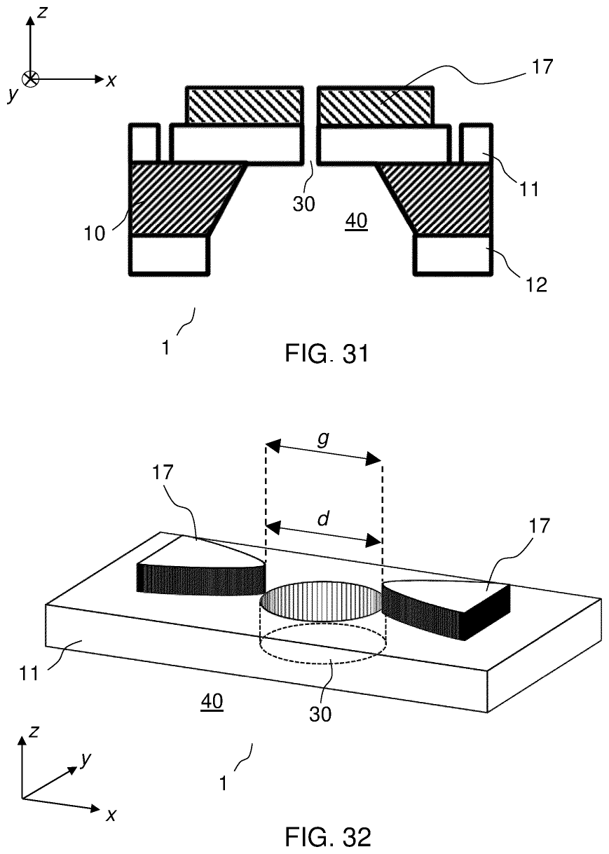

A first aspect of the invention is now described in reference to FIGS. 31-36. This aspect concerns an optical sensing device 1, 1a. Two variants 1, 1a of the device are shown in the accompanying drawings (compare FIGS. 31, 32 and 35, 36). In each case, the device 1, la has a layer structure, which notably comprises a substrate 10, a dielectric layer 11, and antenna elements 17, 17a.

Initially, the substrate 10 may for example be double side polished silicon (Si) wafer 10, the thickness of which is typically 275, 375, or 525 μm. In the present case, the substrate 10 can undergo several processing steps. It must notably be structured to laterally delimit a cavity 40, see FIGS. 31, 33-35. The cavity 40 is typically formed by a recess defined in the substrate 10; the recess delimits the cavity, laterally.

The dielectric layer 11 extends on top of the substrate 10. The layer 11 forms a membrane 11, which spans the cavity 40. The thickness of layer 11 is typically between 10 and 60 nm, preferably between 15 and 35 nm, e.g., between 18 and 22 nm. It is preferably made of Si₃N₄. Notwithstanding their low thicknesses, such membranes were found to be surprisingly stable, mechanically and chemically speaking, a property that manifestly results from fabrication methods as disclosed herein. Note, the device 1, 1a may for example be embodied as a chip (e.g., a sequencing chip) including several membranes, each spanning a respective cavity, notwithstanding the accompanying drawings, where the depicted devices 1, 1a include a single membrane, for simplicity.

The membrane 11 includes n apertures 30 to the cavity 40. These apertures are defined as through holes through the membrane 11. There is at least one aperture (n≥1). With a single aperture, a device 1, 1a as proposed herein may already be able sense a molecule, in principle. In typically applications, though, there are several apertures (n≥2). For applications such as sequencing applications, one will typically seek to fabricate a device with hundreds to thousands to millions of apertures (n≥100). Such apertures are also referred to as nanopores herein, because of their dimensions, as discussed below.

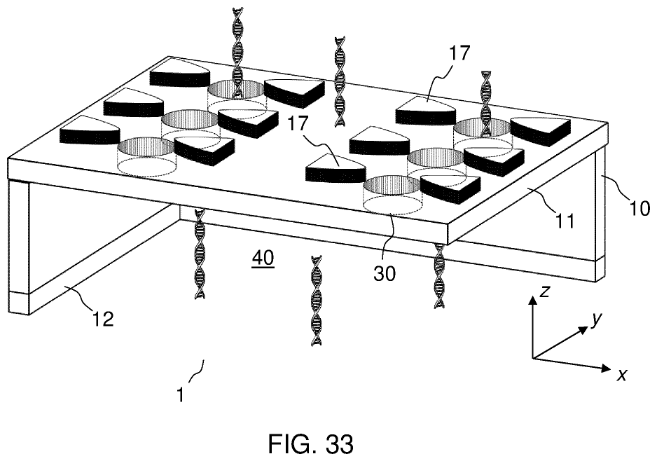

The apertures can for instance be arranged according to a 2D lattice, e.g., a square or rectangular lattice, as assumed in FIG. 33. The average, in-plane separation distance between the apertures will typically be between 1 and 100 microns. In preferred embodiments, the average lattice step is between 1 and 10 microns, preferably between 2 and 7 microns. More preferably, it is between 3 and 6 microns. For example, the average distance between apertures can be of 5 microns. Correspondingly, the areal density of the apertures 30 is between 0.0001 and 1 μm⁻², though preferably between 0.01 and 1 μm⁻². This means that thousands to millions of apertures can be achieved with a chip of, e.g., 5×5 mm². For instance, a low areal density, single membrane structure may already include at least 400 nanopores. Now, a chip may for example include an array of m×m membranes (m≥2, e.g., m=3), each including 400 nanopores or more.

The device 1, 1a further includes n pairs of opposite antenna elements 17, 17a (i.e., plasmonic structures), which are patterned on top of the dielectric layer 11. The antenna elements 17, 17a may typically have a form factor, so as to be asymmetric. Preferably, their largest dimension extends parallel to the plane (x, y). They are preferably made as sharp as possible toward the center, or may exhibit edges, e.g., thanks to frontend surfaces, so as to enhance the electromagnetic hot spot. They can for instance be patterned as bowtie nanostructures, as assumed in the accompanying drawings, although other (asymmetric) shapes can be contemplated.

Remarkably, the antenna elements of each pair are on arranged on opposite lateral sides of the respective apertures 30. In each pair, the antenna elements are separated by a respective gap g. There are n gaps g in total, the gaps extending between opposite antenna elements 17, 17a of respective ones of the n pairs. The gaps extend along respective directions, which are all parallel to a main plane (x, y) of the substrate 10. The pairs of antenna elements 17, 17a will typically be all aligned along parallel directions, as assumed in FIG. 33, where all pairs extend along an axis parallel to direction x. This, however, is a design option.

The n gaps g define, together with the respective apertures, n molecular passages. Each passage extends from the cavity 40, i.e., across the membrane 11, along a direction z that is transverse to the main plane (x, y) of the substrate 10. That is, each passage extends through a respective aperture 30 and a respective gap g.

The average length of the n gaps (as measured along their respective directions) is between 4 nm and 20 nm. Preferably, this dimension is between 4 nm and 15 nm, and more preferably between 5 nm and 12 nm. The gaps separating each antenna element pair are ideally constant, subject to fabrication tolerances (less than or equal to 3 nm, and which can be as low as ±1 to 2 nm, owing to preferred fabrication methods disclosed herein). In fact, both the diameters of the apertures and the gap lengths are preferably essentially constant, subject to a dispersion of less than 2 nm. The structures and their dimensions are checked using Scanning Electron Microscopy (SEM), as well as Transmission Electron Microscopy (TEM). The high resolution permitted by TEM allows to accurately estimate the variability (dispersion) of the dimensions of the nanostructures. Note, the variability (or dispersion) is measured as a standard deviation (namely an uncorrected sample standard deviation).

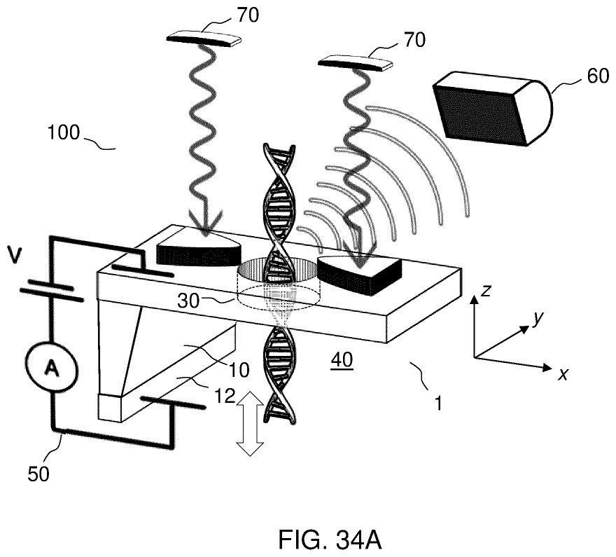

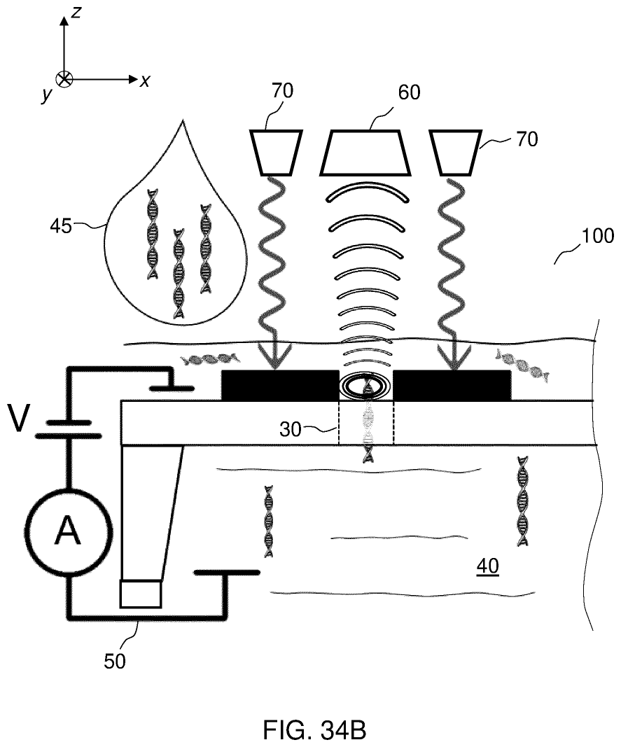

The n gaps define respective electromagnetic field enhancement regions (i.e., hot spot regions). That is, electromagnetic radiation can be concentrated in such regions upon irradiating the antenna elements 17, 17a, with a view to optically sensing analytes (molecules), in operation. Thus, the antenna elements enable an electromagnetic field enhancement mechanism, which can generate a field-enhanced hot spot, wherein electromagnetic radiation can be concentrated. This, in turn, can be exploited to sense an analyte (e.g., a DNA molecule, by surface-enhanced Raman spectroscopy), by irradiating the antenna elements, as known per se. Owing to the dimensions and arrangement of the opposite antenna elements 17, 17a, the resulting electromagnetic field enhancement region is essentially confined to the gap in-between (as illustrated in FIG. 34B), above the apertures.

Moreover, according to the proposed design, the average diameter d of the n apertures 30 is larger than or equal to the average length of the gaps along their respective directions. That is, the average diameter d is larger than or equal to a quantity that is between 4 nm and 20 nm.

The above constraint has important implications, keeping in mind that prior fabrication methods would typically require the patterned antenna element to be laterally recessed with respect to the respective aperture edges. On the contrary, here, the minimal cross-sectional dimension of each molecular passage is limited by a respective gap (as measured along its respective direction), because the antenna elements can be substantially flush with the aperture edge or, even, hang over the aperture. That is, according to the proposed configuration, the dimensions of the electromagnetic field enhancement region are not primarily limited by the aperture dimensions.

As noted above, the inner ends or edge surfaces of the opposite antenna elements 17, 17a may possibly be flush (or substantially flush) with the inner wall delimiting the corresponding apertures 30. That is, the antenna elements 17, 17a can be flush with the apertures along a direction z perpendicular to the main plane (x, y) of the substrate. In variants, the edges of the opposite antenna elements slightly hang over the edge of the respective aperture, in a cantilever fashion. In both cases, the minimal cross-sectional dimension of each passage (as defined by a corresponding aperture in layer 11) is actually determined by the gap between opposite antenna elements. At the limit where the gap length is equal to the aperture diameter, the minimal cross-sectional dimension of each passage is equally limited by each of the aperture diameter and the gap. Still, according to the proposed design, the inner ends of opposite antenna elements 17, 17a will not be laterally recessed (subject to fabrication tolerances, e.g., 1-2 nm) with respect to the edge of the respective aperture, i.e., along an in-plane direction parallel to the main plane of the substrate.

To summarize, the antenna elements 17, 17a can be flush with, or hang over the edges of the aperture underneath, but cannot be laterally recessed with respect to the edges of this aperture. Doing so allows the electromagnetic field enhancement volume to be minimized, for given cross-sectional dimensions of the aperture. This, in turn, allows the electromagnetic field enhancement to be maximized, all things otherwise equal. In particular, large SERS enhancement factors can be achieved, e.g., exceeding 10¹¹.

Obtaining such a configuration is extremely challenging to process with accuracy tolerance on the order of the nm, because the gaps between opposite antenna elements are defined above respective apertures (i.e., the nanopores) defined in a thin membrane. The skilled person will appreciate that the fabrication process is, in that case, much more challenging than in a context where the antenna elements lay flat on a thick substrate. One reason is that the antenna elements and the apertures require distinct process steps, which are incompatible, a priori. In particular, it is not possible to directly etch cylindrical apertures between antenna elements to reach the underlying cavity in a top-down process without damaging the lower end of the apertures in the cavity (i.e., at the level of the top wall surrounding the opening in the cavity) and, in fact, the cavity itself, all the more so if the apertures must have a diameter that is larger than or equal to the gap length, as is the case here. The other way around, it is a priori not possible to process a recess on the backside without damaging apertures in the membrane, should such apertures be processed first.

Fortunately, the present description discloses fabrication methods resolve this incompatibility and further make it possible to achieve nanostructures with a satisfactory accuracy, i.e., on the order of the nm. More generally, the present fabrication methods allow nanostructures to be obtained with deterministic and reproducible shapes, dimensions, and positions, unlike prior fabrication methods.

Still, the proposed fabrication methods rely on common processing techniques, which are compatible with CMOS-technology. It may notably prove advantageous to use CMOS processes in order to transfer signal processing capability much closer to the sensing nanostructures (formed by the antenna pairs and the corresponding apertures). Thus, a spectrometer might be implemented directly in the vicinity of the sensing nanostructures. The present fabrication methods do not require exotic or expensive materials and yet allow the fabrication of unprecedented, deterministic nanostructures, as opposed to randomly created nanostructures, while allowing very good alignment accuracies to be achieved, with high and reproducible field-enhancement for sensing molecules such as RNA and DNA. Thus, ensuring deterministic locations and dimensions of the nanostructures eases the detection.

In particular, the present approach can be used to identify a nucleic acid sequence and can be applied to characterize both single and double stranded DNA sections. In a sequencing device, the apertures play the role of transverse nanopores, through which molecules can pass (also known as tunnelling) thanks to an applied electric field. The electromagnetic field enhancement can further be leveraged to trap molecules at the level of the apertures and, even, control the progression of the molecules stepwise through the nanopores, thanks to the electric field applied concurrently, as in embodiments discussed later.

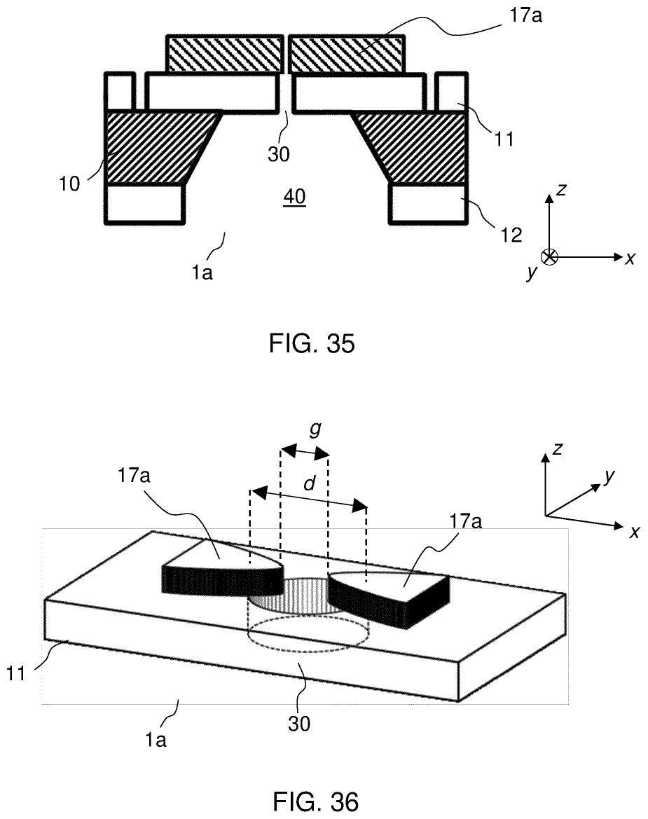

In the example of FIGS. 31-33, the nano-antenna elements are flush with the apertures. More precisely, the optical sensing device 1 is designed in such a way that the average diameter d of the n apertures 30 is substantially equal to the average length of the gaps. I.e., inner ends of the antenna elements 17 of each of the n pairs are substantially flush with inner walls of the respective n apertures 30 in the membrane 11. The equality between the average aperture diameter (in-plane) and the average gap is subject to fabrication tolerances, which, in the present context, can typically be of ±2 nm or less (possibly down to ±1 nm or less, e.g., ±0.8 nm), according to images obtained by SEM and TEM. Achieving flush antenna elements reduces the hot-spot volume and thus increases the electromagnetic radiation concentrations in the gaps, with respect to configurations where the antenna elements are recessed with respect to the apertures. The electromagnetic field enhancement can be further improved by having antenna elements 17a that slightly protrude inwardly, so as to hang over the apertures in a cantilever configuration, as in the device la depicted in FIGS. 35-36. In both cases, the depicted configurations can be practically obtained, thanks to fabrication methods described below. Such configurations allow the electromagnetic radiation concentrations to be optimized, because they make it possible to bring the inner apices of the antenna elements closer together, notwithstanding the apertures and the cavity underneath.

As seen in the accompanying drawings, the present optical sensing devices 1, 1a typically include two dielectric layers 11, 12. That is, the dielectric layer 11 is a first dielectric layer 11 and the device 1, 1a further includes a second dielectric layer 12, which is preferably made of the same material as layer 11 and can have the same thickness. As best seen in FIGS. 31 and 35, the substrate 10 extends between the two dielectric layers 11, 12, on top of the second dielectric layer 12 according to the orientation chosen in the drawings, where the z axis points upwards. As further seen in FIGS. 31 and 35, the substrate 10 and the second dielectric layer 12 can be jointly structured to form a recess, which delimits the cavity 40. In embodiments, the substrate 10 is directly coated by each of the two dielectric layers 11, 12. The first dielectric layer 11 extends on a first side (top side) of the substrate 10, while the second dielectric layer 12 extends on a second side of the substrate, opposite to its first side.

As said, the substrate 10 typically comprises silicon. In variants, however, other substrate materials can be contemplated, such as quartz or glass. Each of the two dielectric layers 11, 12 preferably comprises Si₃N₄, although other compounds such as SiO₂ can be contemplated. Preferred embodiments rely on Si substrates 10 and Si₃N₄ dielectric layers 11, 12. I.e., the substrate 10 is essentially made of Si, while the dielectric layers are essentially made of Si₃N₄. The thickness of each of the first and second dielectric layers 11, 12 is preferably between 10 and 60 nm, and more preferably between 15 and 35 nm. As noted earlier, a 20 nm thick Si₃N₄ membrane 11 was found to be surprisingly stable, mechanically and chemically speaking, for the present purpose.

The antenna elements 17, 17a are preferably made of gold (Au). Other plasmonic materials (typically metals) can be contemplated. However, Au is preferred as it does not corrode, contrary to, e.g., silver, which may oxidize. Aluminum (Al) may potentially be used to fabricate the plasmonic structures acting at shorter wavelengths (i.e., in the UV region of the electromagnetic spectrum). Overall, various materials can be contemplated for the antenna elements, allowing an amplified field-enhancement over a wide energy range, i.e., from ultraviolet (UV) to near IR and full IR.

The antennas (e.g., consisting of gold) are preferably patterned on top of a bonding layer (e.g., including Cr or Ti, though Cr is preferred). In addition, a bonding layer may be used on top of the metallic layer 17, 17a (e.g., Au), in order to improve the adhesion of the EBL resist. Indeed, the extent of the adhesion of the EBL resist directly on top of the metallic layer may depend on the metal.

Besides, the present layer stacks 10-12 may involve additional layers. In particular, the layer structure may include intermediate layers. For example, if a dry etching method is used to open the backside recess to the membrane 11, an intermediate SiOₓ layer can be deposited on top of the Si layer to act as an as an etch stop. As another example, reflector layer structures (dielectric or metallic) may possibly be provided above or below the antennas, and/or laterally around the antenna elements to allow more efficient readouts from the far field or improve coupling of incident, scattered, or emitted light, or increase the sensitivity, through increased photon-antenna interactions. Moreover, any of the layers 10, 11, 12 may possibly be deposited as a stack of several, superimposed layers. Preferred, however, is to rely on single layers of silicon and Si₃N₄, where the Si₃N₄ layers 11, 12 directly coat the substrate 10 (possibly subject to a thin oxide layer surrounding the substrate), for simplicity.

The optical sensing device 1, 1a may further include one or more pairs of electrodes, wherein electrodes of each pair are arranged (e.g., patterned) on opposite sides of the first dielectric layer 11. The aim is to be able to apply a voltage to a liquid in which the device is immersed, in operation, as described later in detail. In variants, such electrodes can be external, i.e., they are not integral with the device 1, 1b and can thus be supplied separately.

Referring to FIGS. 34A and 34B, another aspect of the invention is now described, which concerns an optical sensing apparatus 100. This apparatus includes an optical sensing device 1, 1a such as described above. In the following, this apparatus is assumed to include a device 1 such as shown in FIGS. 31-33 (with flush antenna elements), for the sake of exemplification. However, the apparatus may similarly be based on a device la such as shown in FIGS. 35, 36 (with inwardly protruding antenna elements).

The apparatus 100 further includes a distributed electromagnetic source 70. The source 70, preferably comprises a Laser. For example, the apparatus 100 may be equipped with a microlens array, designed to form beamlets from a large area laser beam, in order to illuminate each antenna pair individually. In all cases, the source 70 is configured to irradiate the antenna elements 17, 17a of each of the n pairs of opposite antenna elements. The aim is to be able to concentrate electromagnetic radiation in the respective electromagnetic field enhancement regions, for optically sensing molecules in the respective gaps g.

Moreover, the apparatus 100 includes a detector 60, which is configured to optically detect optical signals as modulated and/or generated by the molecules in these regions, in operation. The light source and the detector are typically arranged in reflection geometry. The light source 70 is used to optically excite the pairs of antenna elements, while the detector 60 is used to optically detect signals modulated or generated by analytes in the gaps.

This detector 60 may for instance include one or more Raman spectrometers. In preferred embodiments, the detector includes a spatial heterodyne Raman spectrometer (SHRS). An SHRS allows high resolution, broad spectral range, and high throughput, to be simultaneously achieved. Plus, an SHRS does not require scanning. However, an SHRS requires special care with data collection and processing as the measurement is an interferogram, which must be converted to a conventional spectrum.

Although Raman spectroscopy is a preferred characterization technique in the present context, especially in application such as DNA sequencing, it will be apparent to the skilled person that the present devices and apparatuses can also be used with other surface enhanced spectroscopies (e.g., infrared absorption or fluorescence as well as intensity and phase changes based on optical resonance shifts).

The apparatus 100 may further comprises an electrical circuit 50 such as shown in FIGS. 34A and 34B. This circuit 50 notably includes one or more pairs of electrodes on opposite sides of the first dielectric layer 11. The electrical circuit 50 is configured to apply a voltage bias between electrodes of each of the pairs to urge molecules through the passages defined at the apertures and possibly control the progression of the molecules through the passages, as described later in detail, in reference to another aspect of the invention. The circuit 50 should, a minima, allow a constant electric field to be applied, in order to produce a transmembrane bias of a desired voltage difference. The examples of FIGS. 34A and 34B involve very simple electrical circuits, i.e., involving a voltage source, a voltmeter, and an ammeter connected to two electrodes. In practice, however, the circuit may possibly be more sophisticated. For example, the circuit may include multiple electrode pairs, e.g., including one pair for a respective (group of) aperture(s). Such a circuit may notably be used to cause DNA strands to tunnel from the front (top) side of the device (i.e., where the antenna elements are) to the backside (i.e., to the cavity). As noted earlier, the electrodes may possibly be formed integral with the device. In variants, such electrodes are mere end portions of electrical conductors brought in the vicinity of the membrane 11.

A further aspect of the invention is now described in detail (still referring to FIGS. 34A, 34B), which aspect concerns methods for optically sensing an analyte. The main aspects of such methods have already been described, be it implicitly, in reference to the previous aspects of the invention. Such methods rely on an optical sensing device 1, 1a such as described above. Essentially, they consist in irradiating pairs of antenna elements of this device 1, 1a (e.g., thanks to an electromagnetic source 70), to concentrate electromagnetic radiation in the electromagnetic field enhancement regions. The aim is to sense molecules in the gaps by optically detecting optical signals that are modulated and/or generated by the molecules in the gaps, thanks to an optical detector 60. As noted earlier, such a detector may notably include a Raman spectrometer, though it preferably includes a spatial heterodyne Raman spectrometer.

In embodiments, the sensing methods further comprises applying 50 an electric field across the membrane 11, to urge molecules to the passages and trap the molecules at the respective gaps, by virtue of a combined effect of the electric field applied and the electromagnetic radiation concentrated in the respective electromagnetic field enhancement regions. I.e., it is possible to jointly control the irradiation of the antenna elements and the transmembrane electric field to control the progression (i.e., tunnelling) of molecules through the nanopores. It is notably possible to reverse the tunnelling direction, if necessary, and immobilize a molecule (at the level of a given portion thereof) between opposite antenna element apices, for characterizing specific molecule portions or units, such as specific DNA or RNA units, i.e., nucleotides.

In other words, the joint action of the hot spots and electric field can be used to trap the molecules in respective gaps and move the molecules in both directions. E.g., if the measurements obtained are not satisfactory, one may possibly pull back a DNA strand for a certain number of bases and measure the sequence once again.

In general, the device 1, 1a is meant to be immersed in a liquid. In practice, liquid can be pipetted above the membrane 11, as illustrated in FIG. 34B. E.g., a liquid drop of a KCl solution containing DNA molecules can be deposited to top of the dielectric surface 11. As a result, one or more top electrodes of the circuit contact the residual liquid above the layer 11. Meanwhile, the cavity 40 can be filled with a KCl solution too, so as to allow DNA molecules to reach the cavity via the nanopores. The bottom electrode(s) contact(s) the KCl liquid in the cavity. A voltage bias (transmembrane bias) is applied through the electrodes, so as to urge DNA molecules towards the passages and trap them at the gaps, thanks to the combined action of the voltage bias applied and the antenna element irradiation. Meanwhile, the field enhancement enabled by the antenna irradiation allows the trapped molecules to be sensed.

In detail, the electromagnetic field of light incident on a conductor drives the mobile/free charge carriers of the conductor into coherent oscillations, i.e., surface plasmon polaritons (SPP). These oscillations lead to a strong confinement of electromechanical energy near the surface of the conductor, enabling concentration and guiding of light below the diffraction limit. Coupling of SPPs between multiple structures gives rise to extremely high local field enhancements (with local field strengths exceeding 100 times the incident field) in the small gap in between them. The resulting field enhancements (“hot-spot”) are particularly beneficial for effects whose strength increases non-linearly with the field amplitude, such as surface-enhanced fluorescence, infrared absorption, and Raman scattering. The antenna assists both in coupling light into the sensing volume, as well as in transducing the signal towards the detector 60. Specific antenna geometries may further be achieved to help control the direction of the emission. Trapped molecules may for instance scatter incident light, inelastically, at an energy shifted by their vibrational energy, as exploited in Raman spectrometry. In that case, the trapped molecules modulate the incident light. However, other spectrometry principles may possibly be exploited, in which, e.g., the trapped molecule absorb the incident light and reemit photons, which can be detected to characterize the trapped molecules. For example, molecular emissions (fluorescence or phosphorescence) can be detected, whereby molecules in an excited electronic state emit photons and return to a lower-energy state.

That said, preferred applications exploit Raman detection to characterize DNA or RNA molecules. In such case, the device 1, la can be designed as a sequencing chip. The optical signals detected may notably be processed to identify particular nucleic acid sequences, as noted earlier. Note, the present sensing methods can be applied to characterize both single-and double-stranded DNA sections, as well as RNA strands. The same spectroscopic detection methods can further be applied to methylated nucleotides.

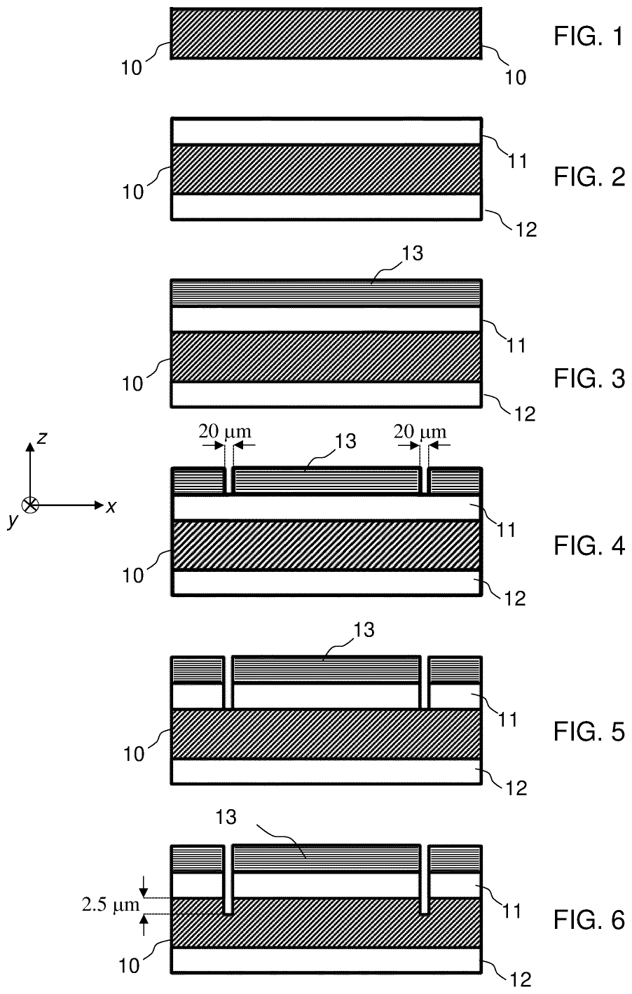

Referring now more specifically to FIGS. 1-31, a final aspect of the invention is described, which concerns methods of fabrication of optical sensing devices 1, 1a such as described earlier. Such methods tackle the formidable challenge of achieving clean nanostructures, where pairs of antenna elements are arranged on opposite lateral sides of nano-apertures formed through a membrane spanning a cavity, while ensuring deterministic and reproducible positions, shapes, and dimensions of the nanostructures. Basically, the proposed fabrication approach revolves around patterning the antenna elements (e.g., using electron beam lithography, or EBL for short), based on an alignment protocol exploiting previously patterned fiducial marks, prior to etching the apertures. However, the apertures are etched only after having protected inner ends of the antenna elements. The recess is then formed on the back side, but only after having coated the upper structures to protect the gaps and apertures. This way, the incompatibility noted earlier can be solved.

In more detail, a substrate 10 is first provided, see FIG. 1. Next, a dielectric layer 11 is deposited on top of the substrate 10, see FIG. 2. Fiducial marks are then patterned on both the dielectric layer 11 and the substrate 10, as illustrated in FIGS. 3-7. Such fiducial marks are preferably patterned using both EBL and a dry etching procedure, as described later in detail in section 2. The fiducial marks may for instance be patterned as slits extending through the dielectric layer 11 and partly in the substrate 10.

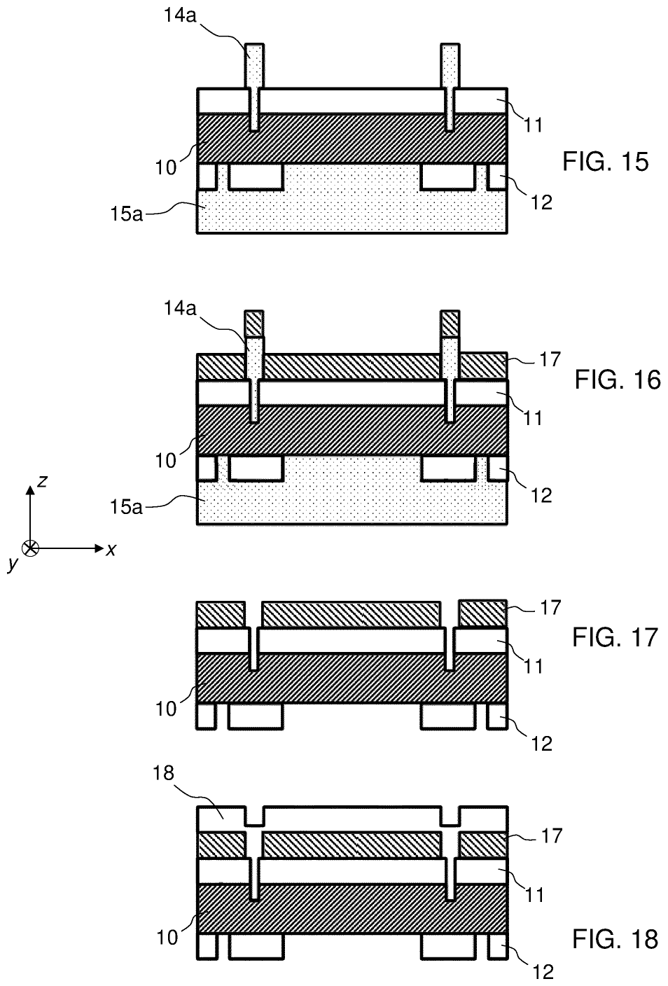

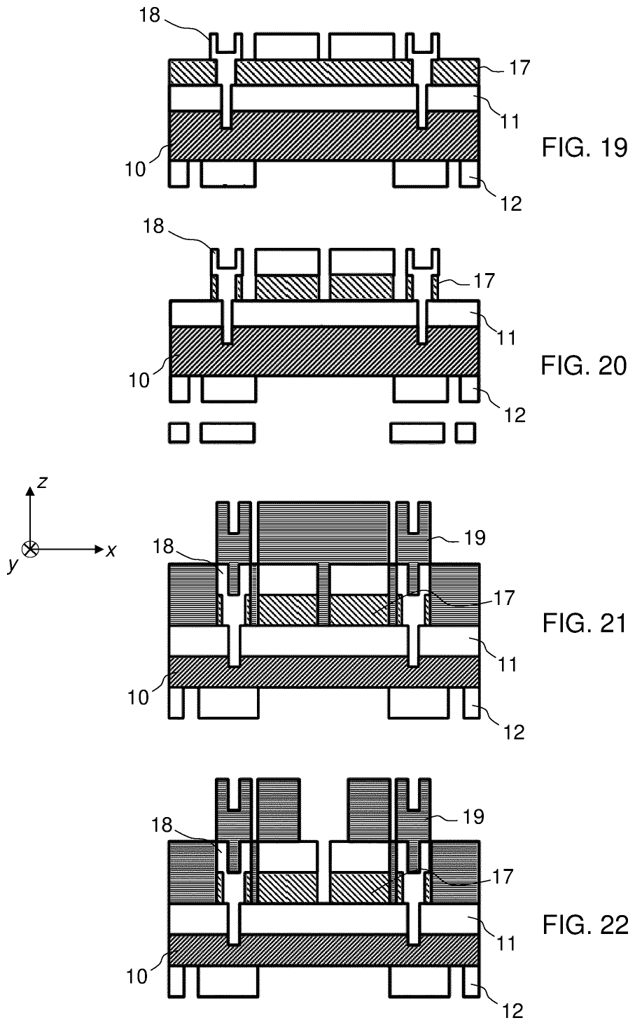

Next, n pairs of laterally opposite antenna elements 17 are patterned on top of the dielectric layer 11, preferably using EBL. Such processing steps are based on an alignment protocol exploiting the fiducial marks previously patterned. The steps are illustrated in FIGS. 13-20. As see in FIGS. 13-19, the pairs of opposite antenna elements 17 are preferably patterned by depositing a photoresist 14a on top of the dielectric layer 11 (see FIG. 13), so as for the photoresist 14a to plug the slits forming the fiducial marks. The aim is to protect the fiducial marks and prevent them from being filled with metal during the subsequent deposition of the metallic layer. Next, the photoresist 14a can be structured by photolithography (FIG. 14), for it to form residual plugs at a level of the slits. A metallic layer 17 is then deposited on top of the dielectric layer 11 and the residual plugs (FIG. 15). The residual plugs are then removed (FIG. 16) to define openings at the level of the slits. An electron beam resist 18 can then be deposited on top of the metallic layer 17. The electron beam resist 18 is then structured by EBL in accordance with the desired shapes for the antenna elements 17, see FIGS. 18, 19. Finally, the metallic layer 17 is etched through the structured electron beam 18 resist, e.g., using ion beam etching, to obtain the antenna elements 17.

This makes it possible to define n respective gaps g extending between the pairs of opposite antenna elements. Each of the n pairs of antenna elements extend along respective directions, which are parallel to the main plane (x, y) of the substrate 10. As discussed earlier, multiple antenna element pairs may possibly be patterned (n>1, preferably n≥100). The average length of the n gaps is between 4 nm and 20 nm.

Importantly, a protective layer 20 is subsequently deposited, for it to coat inner ends of the opposite antenna elements 17 of each pair, as illustrated in FIGS. 21-23. The protective layer 20 may for instance be an alumina layer, which is preferably obtained as follows. First, an electron beam resist 19 is coated. Then, cavities are opened at the level of the gaps. Finally, the protective layer 20 is deposited by atomic layer deposition (ALD), at a temperature that is less than 90 C (to keep it compatible with the materials in layers 17, 19). The deposited layer 20 notably coats the inner ends of the opposite antenna elements 17, see FIGS. 21-23.

Next, the dielectric layer 11 is etched (using a dry etching technique, preferably using reactive-ion etching, or RIE), at locations defined according to the fiducial marks. This is done so as to open n apertures 30 through the dielectric layer 11, between opposite antenna elements 17 of respective pairs. As a result, opposite antenna elements are on opposite lateral sides of respective apertures 30, see FIGS. 24-26.

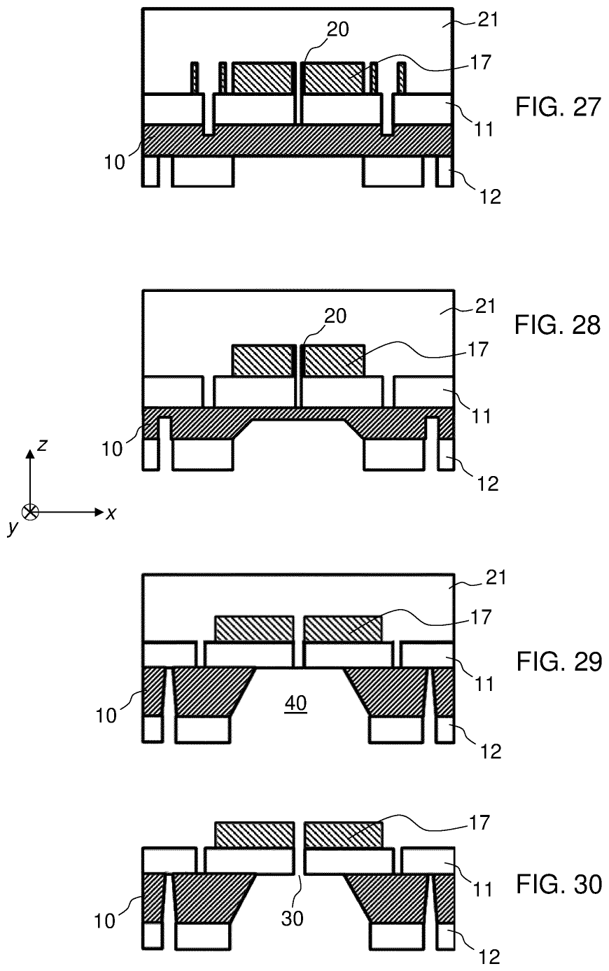

Next, the antenna elements 17 and the dielectric layer 11 are coated with a protective polymer 21, for it to plug the gaps and the apertures 30, see FIG. 27. The substrate 10 can subsequently be structured to form a recess 40 extending up to the dielectric layer 11. As a result, the layer 11 extends on top of residual, peripheral portions of the substrate 10. The layer 11 accordingly forms a membrane 11 spanning the cavity delimited by the recess 40, see FIGS. 28-29.

After that, the protective polymer 21 can finally be removed, to free up n molecular passages. Each passage accordingly extends from the cavity 40, through a respective aperture 30 and between a respective gap g. Each passage extends along a direction z transverse to the main plane (x, y) of the substrate 10, see FIG. 30.

Overall, the above method makes it possible to obtain an optical sensing device 1, in which the n gaps define respective electromagnetic field enhancement regions, as discussed earlier. The control and precision offered by this approach allow clean nanostructures to be obtained, in which the average diameter d of the n apertures 30 is larger than or equal to the average length of the gaps along their respective directions. I.e., the minimal cross-sectional dimension of each passage is limited by the respective gap, not by the aperture diameter (measured parallel to the main plane of the substrate). That is, the electromagnetic enhancement volume can be reduced thanks to the fact that the antenna elements can be made flush with the respective aperture or even hang over the latter. This, in turn, allows the hot spot to be maximized.

There is one backside cavity 40 per membrane and all apertures in that membrane lead to that same cavity. However, the device 1 (e.g., a chip) may also include several membranes extending over respective cavities. That is, every membrane 11 on the chip has its own backside cavity. The backside opening of the recess can notably be achieved via two different processes. A first possibility is to use a KOH etch. This, however, requires that the dimensions of the aperture on the backside are larger than the dimensions of the membrane on the front side, due to anisotropic KOH etching properties. A second possibility is to use deep reactive ion etching (DRIE) through the whole substrate 10 (e.g., a Si wafer). In that case, the dimension of the cavity (recess) on the backside is approximately the same as the membrane. This, in turn, allows the number of membranes on the front side to be increased, since space requirements caused by the KOH etch are reduced significantly. Using DRIE requires an etch stop layer on the front side, between the silicon and the silicon-nitride layer.

As further illustrated in FIGS. 2 and 8-12, the fabrication preferably involves two dielectric layers 11, 12. I.e., a second dielectric layer 12 may be deposited below the substrate 10, see FIG. 2, preferably at the same time as when depositing the first dielectric layer 11, e.g., by low-pressure chemical vapor deposition of Si₃N₄. The second dielectric layer 12 can be patterned after patterning the fiducial marks but prior to patterning the pairs of opposite antenna elements 17, see FIGS. 8-12. This is done so as for residual, peripheral portions of the second dielectric layer 12 to delimit the recess to be formed next.

In preferred embodiments, the substrate 10 essentially comprises silicon, while each of the two dielectric layers 11, 12 essentially comprises Si₃N₄. The antenna elements 17 may for instance essentially comprise Au, as noted earlier. One or more bonding layers may be used, e.g., comprising Cr or Ti. Several variants can be contemplated, in terms of structure and materials. For instance, an etch stop layer (e.g., SiOₓ) may be provided on the front side, next to the dielectric layer 11 on top of the substrate 10, should a dry etching technique be used to form the backside cavity. Plus, reflecting layers may possibly be deposited and structured around the antenna elements, if necessary.

So far, the description of the preferred fabrication methods focusses on flush antenna elements. The following describes how the present fabrication methods can be adapted in order to obtain overhanging configurations such as shown in FIGS. 35 and 36. In such configurations, the antenna elements 17a, 17a protrude inwardly above the hole drawn by the respective aperture 30. An overhanging nanostructure such as shown in FIGS. 35 and 36 can notably be achieved using two different approaches.

The first approach exploits reactive ion etching (RIE) and corresponding sacrificial layers, where such sacrificial layers are made of a material chosen so that an under-etch can be achieved during the RIE step, which results in an undercut. This leads to a configuration where the inward tip of the antenna elements 17a hangs over the nanoaperture 30.

A more sophisticated approach is to use gray-scale electron beam lithography, where a positive resist is employed and a respective, spatially-variable dose is employed during the EBL step so that a 3-dimensional structure made of the positive e-beam resist results. Eventually, the metal layer 17a (e.g., Au) is fabricated using physical vapor deposition (PVD).

The above embodiments have been succinctly described in reference to the accompanying drawings and may accommodate a number of variants. Several combinations of the above features may be contemplated. Examples are given in the next section.

### 2. Specific Embodiments and Technical Implementation Details

### 2.1 Preferred Applications, General Remarks, and Advantages

Particularly preferred embodiments aim at providing a plasmonic solid-state nanopore DNA sequencing chip, adapted for sequencing genetic information, e.g., of human beings, animals, organisms, viruses, bacteria, plants, or fungi. The sequencing chip allows optical signals to be detected and processed to characterize nucleotides and DNA sequence information. Suitable software is used to extract the nucleotide information from Raman spectra measurements. In particular, application can be made to the Raman characterization of each of the four components of DNA, namely adenine, guanine, cytosine, and thymine. RNA molecules can similarly be characterized, as well as uracil, for example.

The proposed approach enables a revolutionary DNA sequencing technology, which increases the throughput of DNA sequencing to unprecedented levels. The present sequencing chips can be leveraged to sequence the whole human genome of millions of people within weeks, instead of months (if not years) as currently allowed by existing sequencing methods. In addition, the proposed approach allows significantly reduced costs since chemistry-based steps are virtually eliminated. It further yields a much better accuracy of the identified genetic information. The proposed technology will also be able to detect methylated DNA. Thanks to the present approach, no special measures are necessary in order to detect methylated nucleotides along the DNA helical strand. The present solutions may notably benefit to research institutions and companies that use DNA sequencing, e.g., for research purposes or to offer DNA sequencing as a service, in particular during periods of pandemic crises, of which COVID-19 is a most striking example.

The present solutions may further benefit to individuals who need or wish to obtain original genetic information. Sequencing an individual's DNA is routinely needed. Now, the present approach can greatly reduce the costs for obtaining an individual's original DNA, with high accuracy. Of particular advantage is the possibility to detect methylated nucleotides, which is notably important in oncology.

The present devices, apparatuses, nanofabrication processes, and sensing methods, allow a very precise manipulation of individual molecules, such as DNA strands that controllably tunnel through nanopores. Due to the deterministic fabrication and placement of the plasmonic structures relative to the nanopores, it is possible to design a highly efficient, massively parallel Raman spectrometer that is capable of concomitantly recording Raman spectra originating from all nanopores in the sequencing chip.

The deterministic and reproducible placement of the plasmonic structures relative to the nanopore relies on advanced alignment procedures offered by the EBL machine employed for the fabrication of the structures. An example of full fabrication sequence is described in the next section.

### 2.2 Preferred Fabrication Method

FIG. 1: the process starts with a double side polished (DSP) silicon (Si) wafer 10. The DSP thickness is preferably of 380 μm. A SiOx layer (not shown) is grown on both sides. This layer will not affect the properties of the resulting chip. However, the SiOx layer will serve as an etch stop when opening the back side via dry etching.

FIG. 2: After a standard RCA cleaning, a low-stress Si₃N₄ layer 11, 12 is deposited on both sides of the Si wafer 10, by low-pressure chemical vapor deposition (LPCVD). The thickness of the dielectric layers 11, 12 obtained is typically between 10 and 50 nm, preferably of about 20 nm.

FIG. 3: A 400 nm thick photoresist layer 13 (CSAR62, electron beam resist) is deposited on top of the first dielectric layer 11.

FIG. 4: 20 μm wide slits are formed by EBL across the thickness of the CSAR62 layer 13. The slits will serve as alignment markers.

FIG. 5: A dry etching process is used to etch the slits further through the top Si₃N₄ layer 11.

FIG. 6: A further dry etching process is used to deepen the slits through the silicon layer 10. The resulting, additional depth will typically be between 1.2 and 2.5 μm. Again, the drawings are not to scale.

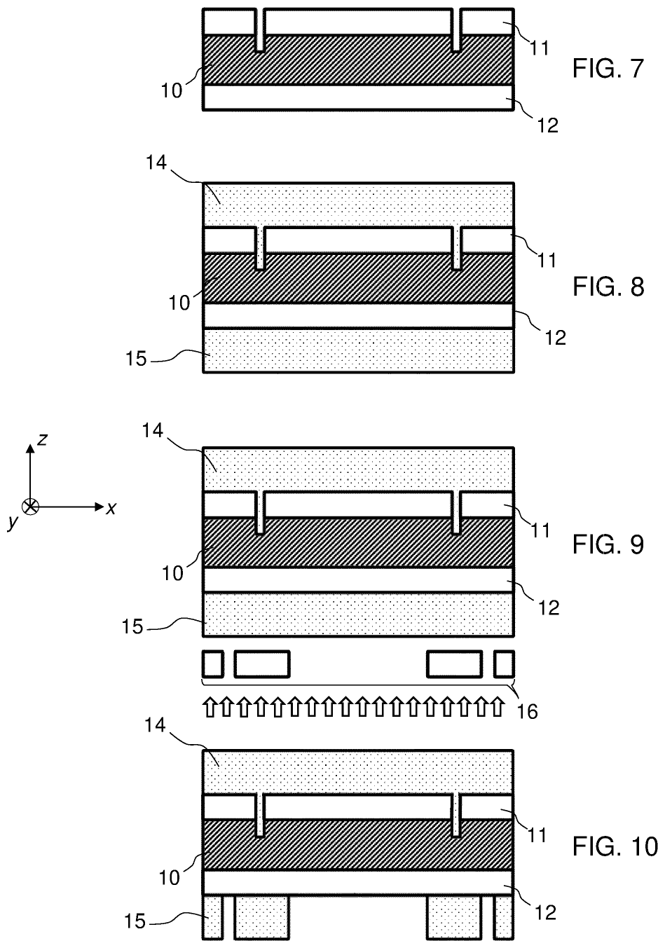

FIG. 7: The CSAR62 layer 13 is stripped.

FIG. 8: The resulting layer structure 10-12 is subject to an HMDS treatment and coated with a photoresist 14, 15 (preferably AZ10XT-20, as assumed in the following, or an equivalent, typically 4 μm thick) on both sides. Note, the front-side 15 is coated first; the exposed side 15 should be baked only once.

FIG. 9: The layer 15 is then exposed to UV light (photolithography) through a structured mask 16, the critical dimension of which is of about 5 μm.

FIG. 10: After development, a structured layer 15 is obtained, which delineate the basis of the upcoming cavity 40. Mind the lower lateral slits formed laterally around the cavity 40, to later be able to singulate the chips. The layer 15 is rinsed with deionized water after development.

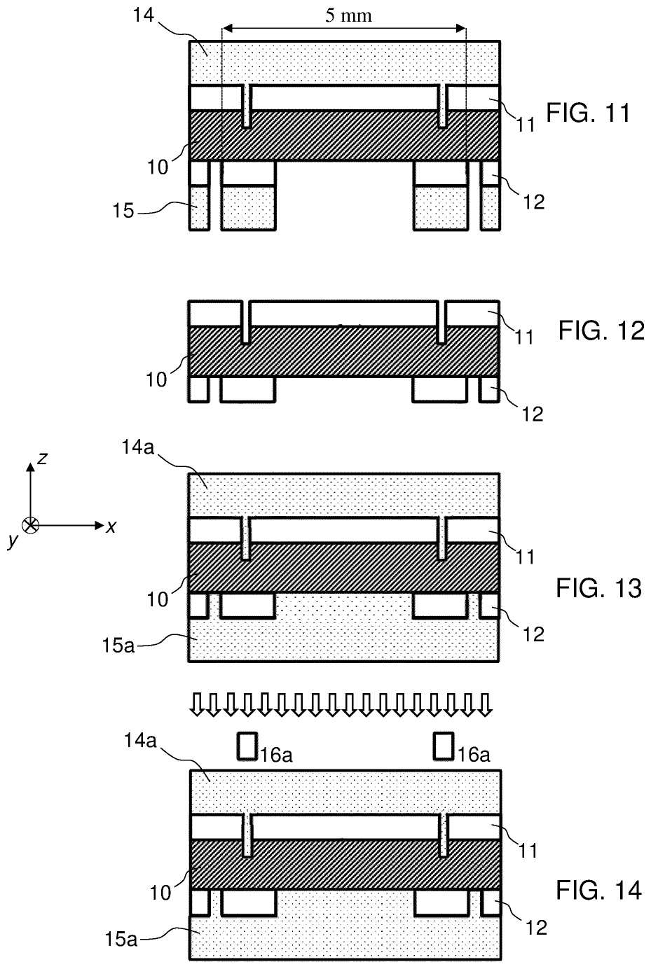

FIG. 11: The lower dielectric (Si₃N₄) layer 12 is then dry etched to deepen the lateral slits into the Si layer 10. The slits accordingly delimit a×a chips, where a is typically between 2 and 5 mm.

FIG. 12: The photoresist 14, 15 (AZ10XT-20) are then stripped.

FIG. 13: The residual layer structure is again subject to an HMDS treatment and coated anew with photoresist layers 14a, 15a (e.g., AZ10XT-20) on both sides, following the same protocol as in FIG. 8, except that the exposed side is now meant to be the top side (corresponding to layer 14a).

FIG. 14: The layer 14a is then exposed to UV light (photolithography) through a structured mask 16a, a critical dimension of which is 5 μm.

FIG. 15: After development, the layer 14a is rinsed with deionized water. Residual structures remain in layer 14a, which will delimit upcoming markers for depositing the antenna elements 17.

FIG. 16: A Cr/Au layer is subsequently evaporated on top of layers 11 (top Si₃N₄ layer) and the residual structures of layer 14a. The Cr layer thickness is of approximately 2 nm, while the Au layer is preferably in the range between 20-50 nm (e.g., about 30 nm). The Cr layer acts as bonding layer.

FIG. 17: The residual parts of layers 14a, 15a (AZ10XT-20) are lifted-off, prior to rinsing with deionized water and isopropyl alcohol (isopropanol).

FIG. 18: An electron beam resist 18 (hydrogen silsesquioxane, or HSQ) is coated on top of the Cr/Au layer 17. The thickness of layer 18 is approximately of 40 nm.

FIG. 19: The top HSQ layer 18 is then patterned through EBL, thereby forming protruding trenches on top of the former slits. Note, the residual portions at the center are shaped according to the antenna elements 17 to be processed next.

FIG. 20: The Au layer 17 is then etched to form the antenna elements 17 of each pair (with a residual gap in-between) using ion beam etching, in accordance with the residual pattern defined in layer 18.

FIG. 21: A ca. 60-100 thick electron beam resist 19 (e.g., CSAR62) is then coated on top of layers 11, 18.

FIG. 22: The resist 19 (CSAR62) is then structured by EBL and rinsed, to clear an area about the gap between the antenna elements 17.

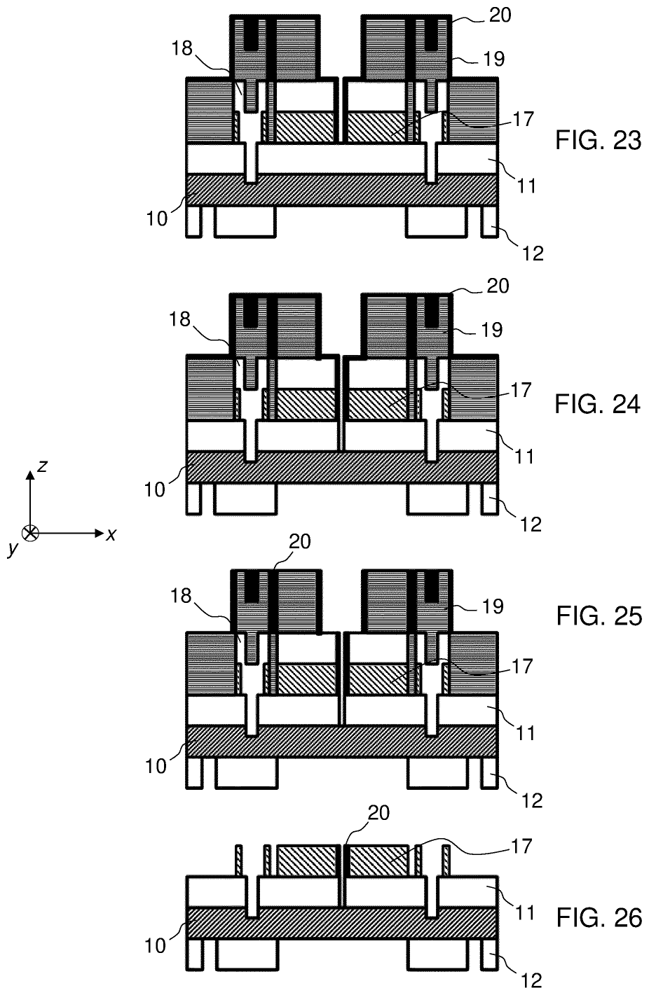

FIG. 23: A protective alumina (Al₂O₃) layer 20 is then deposited (e.g., with a thickness in the range of 5 to 20 nm) by atomic layer deposition (ALD) at a low temperature (approximately 80 C), to keep it compatible with the materials in layers 17, 19.

FIG. 24: Thanks to the protective alumina layer 20, the top dielectric layer 11 can now be dry-etched through its entire thickness to define the apertures 30, using a dry etching technique such as reactive-ion etching (RIE), as assumed in the following.

FIG. 25: The exposed parts of the alumina layer 20 are then wet etched using a buffered oxide etch (BHF) solution. Inner parts of layer 20 remain.

FIG. 26: The resist 19 (CSAR62) layer is subsequently stripped, removing most residual parts of the alumina layer 20 but the parts on inner ends of the antenna elements 17.

FIG. 27: The top side of the chip is then coated with a 6 μm thick ProTEK polymer 21 (a primer, not shown, may have to be coated first), to protect the upper structures.

FIG. 28: The back side of the structure is subsequently etched, first using KOH, to a depth of ca. 360 μm, whereby a residual layer of silicon remains (the recess defining the cavity is not fully formed yet).

FIG. 29: The residual (approximately 20 μm thick) Si layer is carefully etched using XeF₂ on the back side, to ensure a precise control over the etching, in order to reach the lower dielectric (Si₃N₄) layer. The aperture is still clogged with the ProTEK material 21 at this point.

FIG. 30: The ProTEK layer 21 is subsequently stripped, to free up the apertures 30 and gaps g, which removes residual portion of the alumina layer 20 along the inner ends of the antenna elements 17.

FIG. 31: Individual sequencing chips (5 by 5 mm) are eventually singulated from the wafer by breaking at the level of the lower lateral slits.

While the present invention has been described with reference to a limited number of embodiments, variants, and the accompanying drawings, it will be understood by those skilled in the art that various changes may be made, and equivalents may be substituted without departing from the scope of the present invention. In particular, a feature (device-like or method-like) recited in a given embodiment, variant or shown in a drawing may be combined with or replace another feature in another embodiment, variants, or drawing, without departing from the scope of the present invention. Various combinations of the features described in respect of any of the above embodiments or variants may accordingly be contemplated, that remain within the scope of the appended claims. In addition, many minor modifications may be made to adapt a particular situation or material to the teachings of the present invention without departing from its scope. Therefore, it is intended that the present invention not be limited to the particular embodiments disclosed, but that the present invention will include all embodiments falling within the scope of the appended claims. In addition, many other variants than explicitly touched above can be contemplated. For example, other materials and dimensions than those explicitly indicated may be contemplated.

### REFERENCE LIST

1, 1a Optical sensing devices
10 Structured substrate (e.g., Si, forming a recess)
11 First dielectric layer (membrane, e.g., Si3N4)
12 Second dielectric layer (e.g., Si3N4)
13 Thick photoresist layer (e.g., CSAR62)
14, 14a Photoresist (e.g., AZ10XT-20)
15, 15a Photoresist (e.g., AZ10XT-20)
16 Structured mask
16a Structured mask
17 Metallic layer (e.g., Au)
17, 17a Antenna elements
18 Electron beam resist (e.g., HSQ)
19 Electron beam resist (e.g., CSAR62)
20 Protective alumina (Al2O3) layer
21 Protective polymer (e.g., ProTEK)
30 Apertures
40 Cavity
45 Liquid droplet (e.g., KCl solution containing DNA molecules)
50 Electrical circuit
60 Detector (e.g., spatial heterodyne Raman spectrometer)
70 Distributed electromagnetic source (e.g., including laser and microlens array)
100 Optical sensing apparatus
d Aperture diameter
g Gaps between opposite antenna elements

1. An optical sensing device having a layer structure comprising:
a substrate structured to laterally delimit a cavity;
a dielectric layer, which extends on top of the substrate and forms a membrane spanning the cavity, the membrane including n apertures to the cavity, where n≥1; and
n pairs of opposite antenna elements, which are patterned on top of the dielectric layer, on opposite lateral sides of respective ones of the n apertures, and define n respective gaps extending between opposite antenna elements of the n pairs along respective directions parallel to a main plane of the substrate, so as to define n molecular passages, each extending from the cavity through a respective one of the n apertures and a respective one of the n gaps along a direction transverse to the main plane of the substrate,
wherein,
an average length of the n gaps along said respective directions is between 4 nm and 20 nm, whereby the n gaps define respective electromagnetic field enhancement regions, in which electromagnetic radiation can be concentrated upon irradiating the antenna elements, for optically sensing molecules, in operation, and
an average diameter of the n apertures is larger than or equal to said average length of the gaps along said respective directions, whereby a minimal cross-sectional dimension of each of the n molecular passages is limited by a respective one of the n gaps along said respective directions.

2. The optical sensing device according to claim 1, wherein
the number n of the apertures is larger than or equal to 100.

3. The optical sensing device according to claim 2, wherein
the average diameter of the n apertures is equal to the average length of the gaps, subject to ±2 nm, whereby inner ends of the antenna elements of each of the n pairs are substantially flush with inner walls of the respective n apertures in the membrane.

4. The optical sensing device according to claim 2, wherein
the diameters of the apertures and the lengths of the gaps are essentially constant, subject to a dispersion of less than 2 nm.

5. The optical sensing device according to claim 2, wherein
an average, in-plane separation distance between the apertures is between 1 and 10 microns.

6. The optical sensing device according to claim 1, wherein
said dielectric layer is a first dielectric layer,
the optical sensing device includes a second dielectric layer,
the substrate is on top of the second dielectric layer, and
the substrate and the second dielectric layer are jointly structured to form a recess delimiting said cavity.

7. The optical sensing device according to claim 6, wherein
the substrate comprises silicon,
each of the two dielectric layers comprises Si₃N₄, and
each of the antenna elements essentially comprises Au.

8. The optical sensing device according to claim 6, further comprising
one or more pairs of electrodes, wherein the electrodes of each of the pairs are on opposite sides of the first dielectric layer.

9. An optical sensing apparatus, wherein the optical sensing apparatus comprises
an optical sensing device according to claim 1,
a distributed electromagnetic source configured to irradiate the antenna elements of each of the n pairs of opposite antenna elements, so as to concentrate electromagnetic radiation in the respective electromagnetic field enhancement regions, for optically sensing molecules in the respective gaps, and
a detector configured to optically detect optical signals as modulated and/or generated by the molecules in said regions, in operation.

10. The optical sensing apparatus according to claim 9, wherein
the optical sensing apparatus further comprises an electrical circuit comprising one or more pairs of electrodes, wherein the electrodes of each of the pairs are on opposite sides of the first dielectric layer, the electrical circuit configured to apply a voltage bias between electrodes of each of the pairs to urge molecules through the molecular passages.

11. A method of fabrication of an optical sensing device according to claim 1, wherein the method comprises
providing a substrate;
depositing a dielectric layer on top of the substrate;
patterning fiducial marks on both the dielectric layer and the substrate;
patterning n pairs of opposite antenna elements on top of the dielectric layer, based on an alignment protocol exploiting the fiducial marks patterned, so as to define n respective gaps extending between opposite antenna elements of respective ones of the n pairs along respective directions parallel to a main plane of the substrate, wherein n≥1, and an average length of the n gaps along said respective directions is between 4 nm and 20 nm;
depositing a protective layer, for it to coat inner ends of the opposite antenna elements of each of the n pairs;
dry etching the dielectric layer at locations defined according to the fiducial marks, to open n apertures through the dielectric layer, between opposite antenna elements of respective ones of the n pairs, for the opposite antenna elements of the n pairs to be on opposite lateral sides of respective ones of the n apertures;
coating the opposite antenna elements of the n pairs of opposite antenna elements and the dielectric layer with a protective polymer, for it to plug the n gaps and the n apertures;
structuring the substrate to form a recess extending up to the dielectric layer, so as for the latter to extend on top of residual, peripheral portions of the substrate and form a membrane spanning a cavity delimited by the recess, and
removing the protective polymer, to free up n molecular passages, each extending from the cavity through a respective one of the n apertures and a respective one of the n gaps along a direction transverse to the main plane of the substrate, to obtain an optical sensing device, in which
the n gaps define respective electromagnetic field enhancement regions, in which electromagnetic radiation can be concentrated upon irradiating the antenna elements, for optically sensing molecules, in operation of the optical sensing device, and
an average diameter of the n apertures is larger than or equal to said average length of the gaps along said respective directions, whereby a minimal cross-sectional dimension of each of the n molecular passages is limited by a respective one of the n gaps along said respective directions.

12. The method according to claim 11, wherein
the dielectric layer is a first dielectric layer; and
the method further comprises
depositing a second dielectric layer below the substrate; and
after patterning the fiducial marks and prior to patterning the n pairs of opposite antenna elements, patterning the second dielectric layer, so as for residual, peripheral portions of the second dielectric layer to delimit the recess to be formed next.

13. The method according to claim 11, wherein
the fiducial marks are patterned using electron beam lithography and a dry etching procedure.

14. The method according to claim 13, wherein
the fiducial marks are patterned as slits extending through the dielectric layer and partly in the substrate.

15. The method according to claim 14, wherein the n pairs of opposite antenna elements are patterned by:
depositing a photoresist on top of the dielectric layer, for the photoresist to plug the slits;
structuring the photoresist by photolithography, for it to form residual plugs at a level of the slits;
depositing a metallic layer on top of the dielectric layer and the residual plugs, and removing the residual plugs to define openings at the level of the slits;
depositing an electron beam resist on top of the metallic layer and structuring the electron beam resist by electron beam lithography in accordance with shapes of the antenna elements; and
etching the metallic layer through the structured electron beam resist using ion beam etching, to obtain the antenna elements.

16. The method according to claim 11, wherein
the protective layer is an alumina layer, which is obtained by
coating an electron beam resist,
opening cavities at the level of the gaps, and
depositing the protective layer by atomic layer deposition at a temperature that is less than 90 C, for it to notably coat the inner ends of the opposite antenna elements of each of the n pairs.

17. The method according to claim 11, wherein
the substrate essentially comprises silicon, each of the two dielectric layers essentially comprises Si₃N₄, and each of the antenna elements essentially comprises Au.

18. A method for optically sensing an analyte, the method comprising:
providing an optical sensing device having a layer structure comprising:
a substrate structured to laterally delimit a cavity;
a dielectric layer, which extends on top of the substrate and forms a membrane spanning the cavity, the membrane including n apertures to the cavity, where n≥1; and
n pairs of opposite antenna elements, which are patterned on top of the dielectric layer, on opposite lateral sides of respective ones of the n apertures, and define n respective gaps extending between opposite antenna elements of the n pairs along respective directions parallel to a main plane of the substrate, so as to define n molecular passages, each extending from the cavity through a respective one of the n apertures and a respective one of the n gaps along a direction transverse to the main plane of the substrate, wherein,
an average length of the n gaps along said respective directions is between 4 nm and 20 nm, whereby the n gaps define respective electromagnetic field enhancement regions, in which electromagnetic radiation can be concentrated upon irradiating the antenna elements, for optically sensing molecules, in operation, and
an average diameter of the n apertures is larger than or equal to said average length of the gaps along said respective directions, whereby a minimal cross-sectional dimension of each of the n molecular passages is limited by a respective one of the n gaps along said respective directions,
irradiating the pairs of antenna elements of the optical sensing device, to concentrate electromagnetic radiation in the electromagnetic field enhancement regions, and
sensing molecules in the n gaps by optically detecting optical signals that are modulated and/or generated by the molecules in the n gaps, thanks to an optical detector.

19. The method according to claim 18, wherein the method further comprises
applying an electric field across the membrane to urge molecules to the molecular passages and trap the molecules at the respective gaps, by virtue of a combined effect of the electric field applied and the electromagnetic radiation concentrated in the respective electromagnetic field enhancement regions.

20. The method according to claim 18, wherein the method further comprises:
jointly controlling the applied electric field and an intensity at which the pairs of antenna elements are irradiated to control a progression of the molecules through the n molecular passages, while optically detecting the optical signals.

21-22. (canceled)
